(19) United States
(12) Patent Application Publication
(10) Pub. No.: US 2014/0073969 A1
(43) Pub. Date: Mar. 13, 2014

(54) MOBILE CARDIAC HEALTH MONITORING

(71) Applicant: NeuroSky, Inc., San Jose, CA (US)

(72) Inventors: Rui Zou, Sunnyvale, CA (US); An Luo, San Jose, CA (US); Cheng-I Chuang, Saratoga, CA (US)

(21) Appl. No.: 13/973,916

(22) Filed: Aug. 22, 2013

Related U.S. Application Data
(60) Provisional application No. 61/700,260, filed on Sep. 12, 2012.

(51) Int. Cl. A61B 5/00; A61B 5/021; A61B 5/0404; A61B 5/026; A61B 5/04; A61B 5/0456; A61B 5/029

(52) U.S. Cl. CPC A61B 5/6898; A61B 5/0456; A61B 5/02125; A61B 5/029; A61B 5/0261; A61B 5/04012; A61B 5/0404; A61B 2560/0468
USPC 600/479

(57) ABSTRACT

Techniques for mobile cardiac health monitoring are disclosed. In some embodiments, a system for mobile cardiac health monitoring includes a mobile device that includes a processor configured to receive a first set of data from an optical sensor; receive a second set of data from an electrical sensor; and perform a plurality of cardiac health measurements using the first set of data from the optical sensor and the second set of data from the electrical sensor.

### CROSS REFERENCE TO OTHER APPLICATIONS

This application claims priority to U.S. Provisional Patent Application No. 61/700,260 (Attorney Docket No. NEURP018+) entitled MOBILE CARDIAC HEALTH MONITORING filed Sep. 12, 2012, which is incorporated herein by reference for all purposes.

### BACKGROUND OF THE INVENTION

According to the Center for Disease Control and Prevention, heart disease is the leading cause of death in the United States, which is responsible for one among every three deaths in the United States. For example, there are approximately 2,000,000 heart attacks and strokes that occur in the United States every year, which costs the United States an estimated $444 billion/year in health care costs. Unfortunately, nearly 15% of people at risk for cardiovascular disease are undiagnosed and less likely to take preventive action.

### BRIEF DESCRIPTION OF THE DRAWINGS

Various embodiments of the invention are disclosed in the following detailed description and the accompanying drawings.

FIG. 1A shows a front view of a mobile cardiac health monitoring system using a smart phone in a case in accordance with some embodiments.

FIG. 1B shows a back view of a mobile cardiac health monitoring system using a smart phone in a case in accordance with some embodiments.

FIG. 2 is a functional block diagram illustrating a configuration of a mobile device that performs mobile cardiac health monitoring in accordance with some embodiments.

FIG. 3 shows a view illustrating how to measure electrocardiography (ECG) and pulse wave of a user using a mobile device that performs mobile cardiac health monitoring in accordance with some embodiments.

FIG. 4 shows an ECG waveform detected by an ECG sensor in accordance with some embodiments.

FIG. 5 shows a pulse wave detected by an optical sensor of a mobile device that performs mobile cardiac health monitoring in accordance with some embodiments.

FIG. 6 shows a Pulse Wave Transit Time (PWTT) measured from an ECG waveform and pulse wave using a mobile device that performs mobile cardiac health monitoring in accordance with some embodiments.

FIG. 7 is a flow diagram for performing mobile cardiac health monitoring in accordance with some embodiments.

FIG. 8 is another flow diagram for performing mobile cardiac health monitoring in accordance with some embodiments.

### DETAILED DESCRIPTION

The invention can be implemented in numerous ways, including as a process; an apparatus; a system; a composition of matter; a computer program product embodied on a computer readable storage medium; and/or a processor, such as a processor configured to execute instructions stored on and/or provided by a memory coupled to the processor. In this specification, these implementations, or any other form that the invention may take, may be referred to as techniques. In general, the order of the steps of disclosed processes may be altered within the scope of the invention. Unless stated otherwise, a component such as a processor or a memory described as being configured to perform a task may be implemented as a general component that is temporarily configured to perform the task at a given time or a specific component that is manufactured to perform the task. As used herein, the term ‘processor’ refers to one or more devices, circuits, and/or processing cores configured to process data, such as computer program instructions.

A detailed description of one or more embodiments of the invention is provided below along with accompanying figures that illustrate the principles of the invention. The invention is described in connection with such embodiments, but the invention is not limited to any embodiment. The scope of the invention is limited only by the claims and the invention encompasses numerous alternatives, modifications and equivalents. Numerous specific details are set forth in the following description in order to provide a thorough understanding of the invention. These details are provided for the purpose of example and the invention may be practiced according to the claims without some or all of these specific details. For the purpose of clarity, technical material that is known in the technical fields related to the invention has not been described in detail so that the invention is not unnecessarily obscured.

Conventional cardiovascular monitoring systems capable of measuring multiple vital signs, such as electrocardiography (ECG) signals, heart rate, respiration, cardiac output, blood oxygen saturation, and blood pressure are used to assess patients' cardiovascular function in operating rooms, intensive care units (ICUs), and patient rooms of hospital facilities. However, such conventional cardiovascular monitoring systems are typically cumbersome and inconvenient, and generally require medical personnel to operate such conventional cardiovascular monitoring systems. Some measurements are invasive, such as cardiac output. Some measurements involve cuffs or finger clips, such as blood pressure and blood oxygen saturation. These limitations of conventional cardiovascular monitoring systems make it incapable and/or impractical for efficiently and effectively monitoring the cardiac health status of individuals in their daily routine to detect, monitor, and/or prevent various heart disorders.

The emergence of mobile technologies and advances in bio-sensors are promising to change the conventional healthcare system, to facilitate systems that provide for mobile and individual-centered healthcare systems. Mobile monitoring systems can provide continuous physiological data and better information regarding the general health of individuals. For example, such a mobile cardiac health monitoring system can reduce health care costs by disease prevention and enhancement of the quality of life with disease management.

Accordingly, a mobile device is disclosed that determines a user's cardiac health status by monitoring multiple key cardiovascular parameters and/or their index, such as ECG, heart rate, cardiac output, and blood pressure in a continuous and non-invasive fashion. For example, users can conveniently carry handheld mobile devices anywhere and conduct self-monitoring whenever desired or necessary (e.g., all the time or as needed or when convenient).

Monitoring the heart activity through ECG is a common technique, performed by placing ECG electrodes to the skin to measure the electrical activity of the heart. Wearable ECG and heart rate monitors have been used to monitor health status and exercise activity. But these devices are limited to measuring one or two parameters. Multi-parameter monitoring techniques as disclosed herein provides a more reliable and useful technique for monitoring cardiac health status compared to single-parameter monitoring.

The continuous, cuff-less and non-invasive measurement of blood pressure is more desirable for people to regularly monitor their blood pressure. Estimation of blood pressure using other physiological parameters has been studied extensively. It is commonly accepted that pulse wave transmit time (PWTT) can be regarded as an index of arterial stiffness, and has been employed as an indirect estimation of blood pressure. PWTT can be measured as the time interval between the R-wave peak of ECG and the pulse wave arrival in the same cardiac cycle, when ECG and pulse wave are simultaneously recorded. PWTT was originally applied in the area of blood pressure estimation by Gribbin et al. in 1976 (see B. Gribbin et al. “Pulse wave velocity as a measure of blood pressure change”, Psychophysiology, vol. 13, no. 1, pp. 86-90, 1976). Since then, researchers have studied the mechanism and feasibility of this method. In 1979, Obrist discussed that PWTT can be used as an index of blood pressure. Lane studied the relationships between PWTT and systolic blood pressure, diastolic blood pressure, and mean arterial blood pressure by experiments in 1983 (see P. A. Obrist, et al. “Pulse transit time: relationship to blood pressure and myocardial performance,” Psychophysiology, vol. 16, no. 3, pp. 292-301, 1979). Different expressions have been derived to characterize the relationship between the blood pressure and the PWTT, such as described in the following paper: M. Y. Wong et al. “An Estimation of the Cuffless Blood Pressure Estimation Based on Pulse Transit Time Techniques: a Half Year Study on Normotensive Subjects”, Cardiovasc Eng. DOI 10.1007/s 10558-009-9070-7.

Studies have shown that PWTT can also be used to estimate another important cardiovascular parameter, cardiac output. Cardiac output generally refers to the total volume of blood pumped by the ventricle per minute. Diseases of the cardiovascular system are often associated with the change in cardiac output, particularly the pandemic diseases of hypertension and heart failure. Presently, cardiac output is usually only monitored on patients in ICUs or operating rooms, because it is typically performed using an invasive measurement involving insertion of a catheter through a pulmonary artery. Studies have shown that an estimate of cardiac output based on PWTT is highly correlated with invasive measurement of cardiac output. Accordingly, as disclosed herein, such a non-invasive technique provides a convenient way for users to trace cardiac output trends on a daily basis.

Pulse wave is usually measured by a pulse oximeter. When measuring pulse wave, a photoplethysmogram (PPG) sensor is typically placed on a fingertip or earlobe to track the pulse traveling from the heart to the peripheral point. Light of two different wavelengths is passed through the patient to a photo detector. The changing absorbance at each of the wavelengths is measured, allowing determination of the absorbance due to the pulsing arterial blood. A recent study, C. G. Scully et al. “Physiological Parameter Monitoring from Optical Recordings With a Mobile Phone” (IEEE Transaction on Biomedical Engineering, Vol. 59, No. 2, 2012), has demonstrated that the color change signals detected by an optical sensor of a mobile phone can be used as an assessment of pulse wave when putting a fingertip on the optical lens of a video camera.

The increasing processing power and sensor functionality of smart phones and mobile devices allow such mobile devices to serve as apparatus for a convenient health care monitor. In some embodiments, a mobile device that includes an electrical sensor(s) (e.g., two ECG sensors can be provided with/integrated with the mobile device and/or a case for the mobile device, in which the ECG sensors can communicate wirelessly with the mobile device through Bluetooth, radio frequency (RF), or other wireless telecommunication techniques) and an optical sensor (e.g., commercially available optical sensors provided with/integrated into commercially available smart phones can be used and configured to implement various techniques as further described herein) is configured to record pulse wave and combine the recorded pulse wave with simultaneous ECG recording captured by an ECG sensor(s) to derive other cardiovascular related information, such as blood pressure and cardiac output related index.

In some embodiments, a handheld mobile device is provided, such as a smart phone, tablet, or laptop that includes an ECG measurement module and an analysis module. In some embodiments, the ECG measurement module is constructed to be detachably coupled with the mobile device, which can be constructed in the form of, for example, a dongle (e.g., or another similar type of external component that can communicate with and/or be coupled with the mobile device) to attach to a mobile device, or in the form of a case to accommodate the mobile device. In some embodiments, the ECG device can be embedded inside a mobile device in the form of a chip or a chip set (e.g., one or more processors). In some embodiments, the ECG measurement module can be constructed as a standalone mobile device, which can communicate with mobile devices through Bluetooth, RF, or other wireless telecommunication techniques.

In some embodiments, the analysis module includes analyzing pulse wave based on the varying images detected by optical sensors, synchronizing pulse wave with simultaneously recorded ECG data, and deriving cardiac output and blood pressure index. In some embodiments, the analysis module is implemented as a software program executed on a central processor of the mobile device. In some embodiments, the ECG sensors are installed at a position on the mobile device with which the user's hand can be in contact with the ECG sensor(s) as well as the optical sensor by placing fingers onto the optical lens of the optical sensor at the same time, when the user is holding the mobile device.

In some embodiments, a handheld mobile cardiac health monitor is provided to track multiple cardiovascular parameters and/or related information, such as ECG, heart rate, blood pressure, and cardiac output. In some embodiments, such information can be used to help evaluate a user's cardiovascular function and its change over time. Thus, a doctor may be able to treat a patient based on such information. For example, the occurrence of a cardiovascular event, such as for example, a heart attack, can be detected if abnormal or sudden changes of cardiovascular parameters are detected or shown.

In some embodiments, an algorithm is embedded in the recording unit and makes decisions in real-time. In some embodiments, the data is transmitted wirelessly to another device or functional element (e.g., a computer or other computing or functional processing device) where the decision is made and proper actions are performed.

In some embodiments, a storage unit, such as on-board memory or a memory card, is provided such that when abnormal parameters are present, such data is recorded continuously for further evaluation. In some embodiments, users can voluntarily and continuously record data (e.g., on such a storage unit).

In some embodiments, a wireless transmission unit is included in the mobile device to trigger an alarm (e.g., to call or notify a caregiver and/or doctor) or send commands. In some embodiments, a GPS element is also included to record/store location information of the user/patient to communicate location information of the user/patient when a cardiovascular disease or a heart attack event is determined, such as using the wireless transmission unit. Once an event, disease, or a heart attack, is detected, a warning is triggered to allow the patient/caregiver/doctor to take appropriate actions. Treatments such as medication can also be given to stop or alleviate the situation.

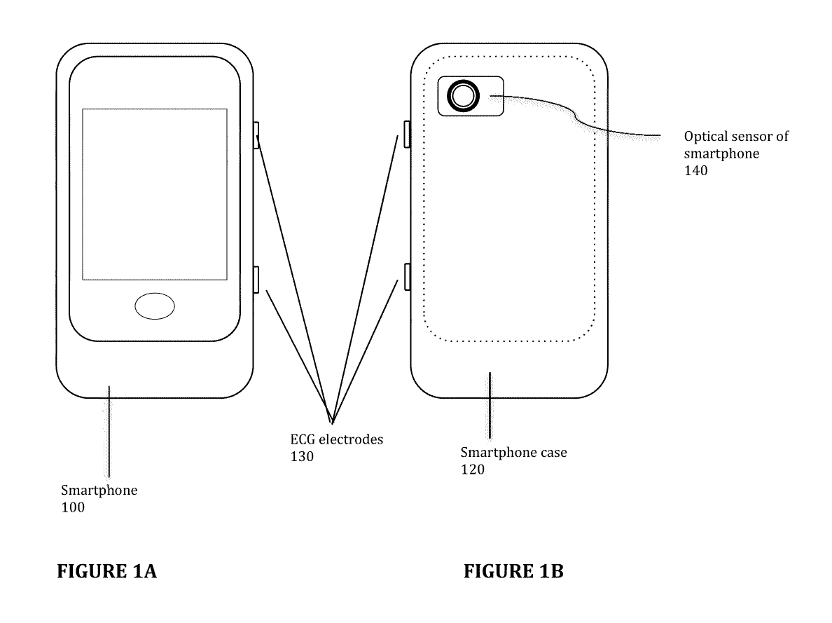

FIG. 1A shows a front view of a mobile cardiac health monitoring system using a smart phone in a case in accordance with some embodiments. FIG. 1B shows a back view of a mobile cardiac health monitoring system using a smart phone in a case in accordance with some embodiments. As shown, a smart phone 100 includes ECG electrodes 130 and an optical sensor 140. As also shown, smart phone 100 is enclosed in smart phone case 120, and ECG electrodes 130 are integrated in smart phone case 120. In some embodiments, ECG electrodes are integrated with smart phone 100. In some implementations, smart phone 100 includes a processor that can be configured to select pixel resolution at a sampling rate (e.g., such as 720×480 pixel resolution at 30 hertz (Hz)) for optical sensor 140 for providing data from the optical sensor for various techniques for mobile cardiac health monitoring as further described herein with respect to various embodiments. In some implement, other types of electrical sensors can be used to perform various techniques for mobile cardiac health monitoring as further described herein with respect to various embodiments.

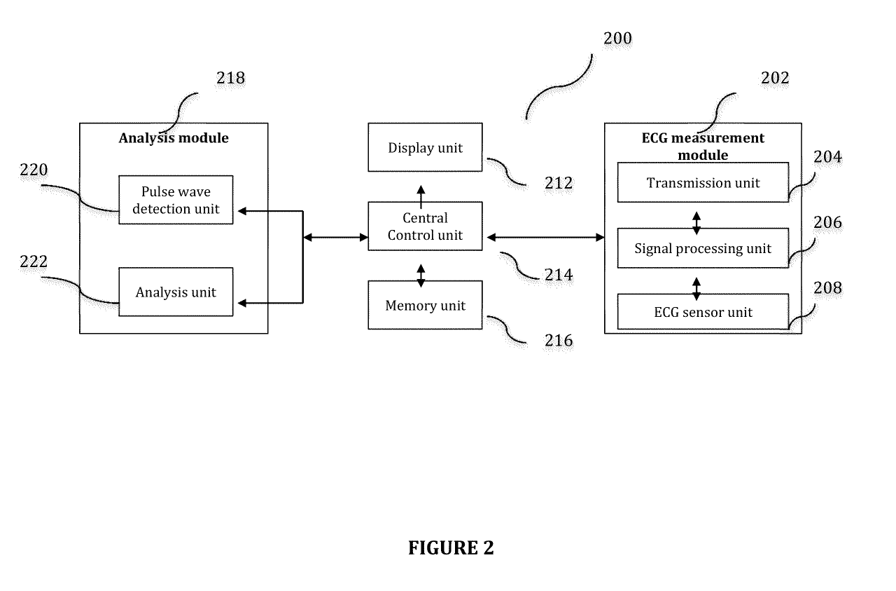

FIG. 2 is a functional block diagram illustrating a configuration of a mobile device that performs mobile cardiac health monitoring in accordance with some embodiments. In particular, FIG. 2 provides a configuration of a mobile device 200 that performs mobile cardiac health monitoring in accordance with some embodiments. As shown, mobile device 200 includes an ECG measurement module 202, a display unit 212, a central control unit 214, a memory unit 216, and an analysis module 218.

As shown in FIG. 2, ECG measurement module 202 includes an ECG sensor unit 208 for detecting ECG from a user, a signal-processing unit 206 to process and analyze ECG and heart rate, and a transmission unit 204 for transmitting data to central control unit 214 of mobile device 200.

Display unit 212 displays ECG and heart rate signals from ECG measurement module 202, as well as the cardiac output and blood pressure estimation from analysis module 218 in, for example, a simultaneous and continuous fashion.

Memory unit 230 stores detected and derived signals for retrospective review and/or further investigation for, for example, medical professionals.

As also shown in FIG. 2, analysis module 218 includes pulse wave detection unit 220 and analysis unit 222. Pulse wave detection unit 220 of analysis module 218 functions to obtain pulse wave data from detecting the varying color signals of a fingertip placed in contact with an optical sensor of the mobile device 200 (e.g., optical sensor 140 as shown with respect to FIG. 1). In some implementations, central control unit 214 can be configured to receive optical data from an optical sensor of the mobile device (e.g., in some case, the central control unit can also configure a desired pixel resolution and sampling rate of the optical sensor, such as 720×480 pixel resolution at 30 hertz (Hz)).

In some implementations, analysis unit 222 of analysis module 218 synchronizes the simultaneous ECG data received from ECG measurement module 202 and pulse wave data received from pulse wave detection unit 220. For example, analysis unit 222 can then use such synchronized ECG data and pulse wave data to measure Pulse Wave Transit Time (PWTT) and can also estimate blood pressure and cardiac output. In some embodiments, analysis module 218 is implemented as a software program executed on central control unit 214 (e.g., a central processor of the mobile device). In some implementations, the analysis module, or certain functional modules of the analysis module, can be implemented in hardware, such as an application-specific integrated circuit (ASIC) or a field-programmable gate array (FPGA).

For example, mobile device 200 can be any of the following or similar portable computing devices, such as a smart phone, tablet computer, and/or laptop computer. Other example mobile devices can include wearable computing devices (e.g., a smart watch, a GPS-enabled watch, a wireless enabled wearable device, and/or other similar types of wearable computing devices) and/or various other mobile computing devices capable of being integrated with an optical sensor and an electrical sensor (e.g., ECG sensor) and/or a case coupled to such a mobile computing device that can be integrated with an optical sensor and an electrical sensor (e.g., ECG sensor).

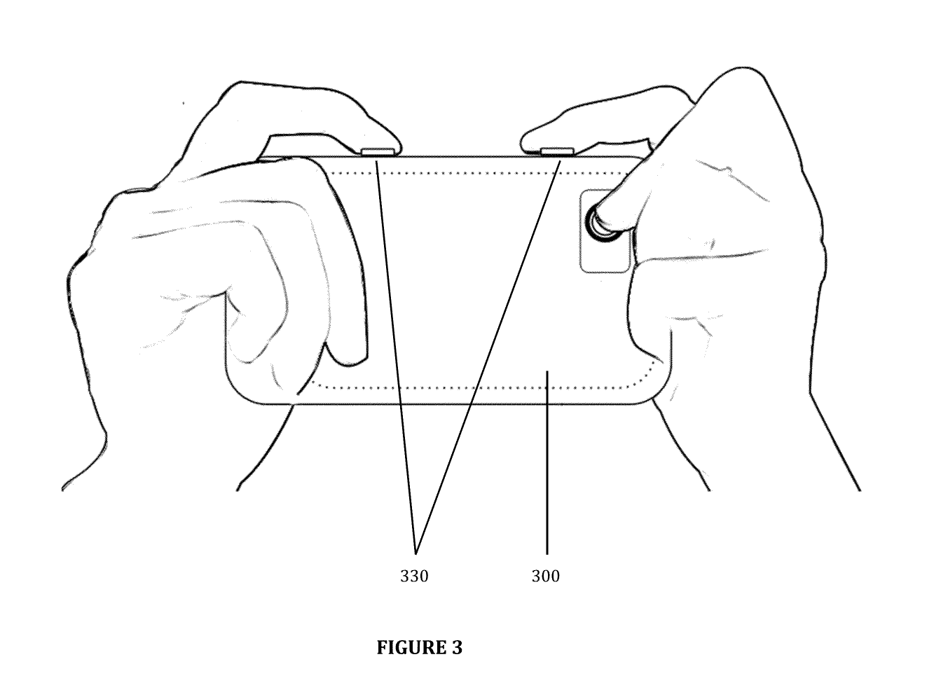

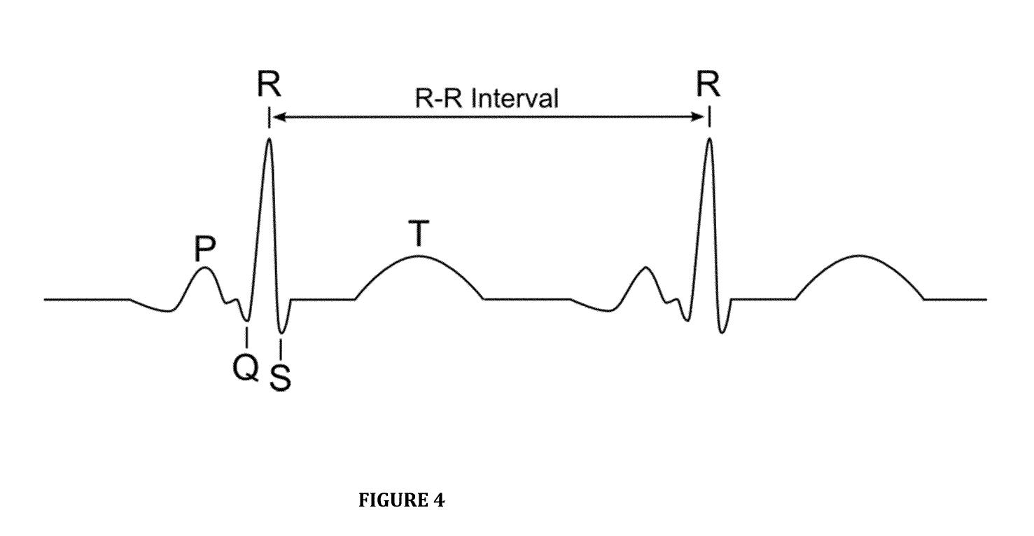

FIG. 3 shows a view illustrating how to measure an ECG and pulse wave of a user using a mobile device that performs mobile cardiac health monitoring in accordance with some embodiments. In particular, FIG. 3 provides a view illustrating how to simultaneously measure ECG and pulse wave using mobile device 300 that includes a case integrated with ECG sensors as shown. In some embodiments and referring back to FIG. 2, ECG measurement module 202 is configured to detachably mount to the mobile device. For example, the module 202 can be configured in the form of a case to accommodate the mobile device 300 as shown in FIG. 4. In some implementations, ECG measurement module 202 can be configured in the form of a dongle attached to the mobile device. As shown in FIG. 3, for example, a user can place a finger of one hand on an optical lens of mobile device 300 and meanwhile place two index/middle fingers of both hands on ECG electrodes 330.

FIG. 4 shows normal features of the ECG detected by an ECG sensor in accordance with some embodiments. ECG records the electrical activity of the heart by detecting the tiny electrical changes using the skin electrodes. The detected ECG waveform data includes P, Q, R, S, and T waves. Each part of ECG waveform has its physical meaning. P wave reflects atrial depolarization (e.g., or contraction). QRS complex reflects the rapid depolarization of ventricles. T wave represents the repolarization (e.g., or recovery) of ventricles. R-R interval illustrates the inter-beat timing.

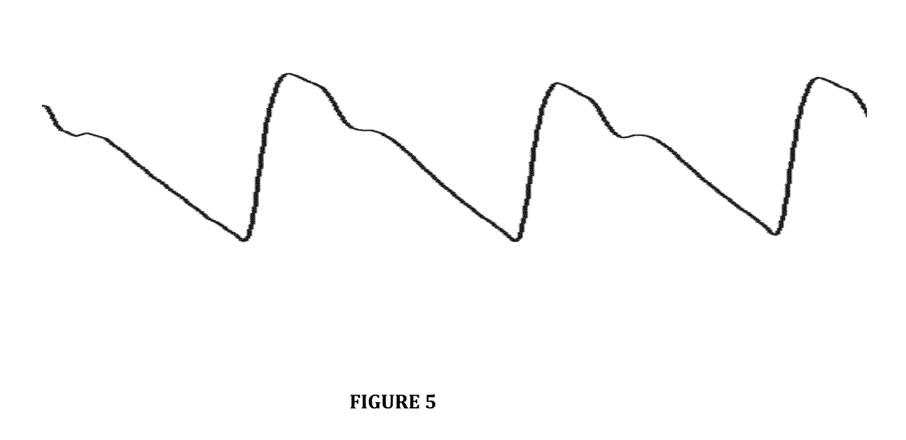

FIG. 5 shows a pulse wave detected by an optical sensor of a mobile device that performs mobile cardiac health monitoring in accordance with some embodiments.

In particular, FIGS. 4 and 5 show an ECG waveform and pulse wave detected and processed, for example, using ECG measurement module 202 and analysis module 218 as shown in and described above with respect to FIG. 2.

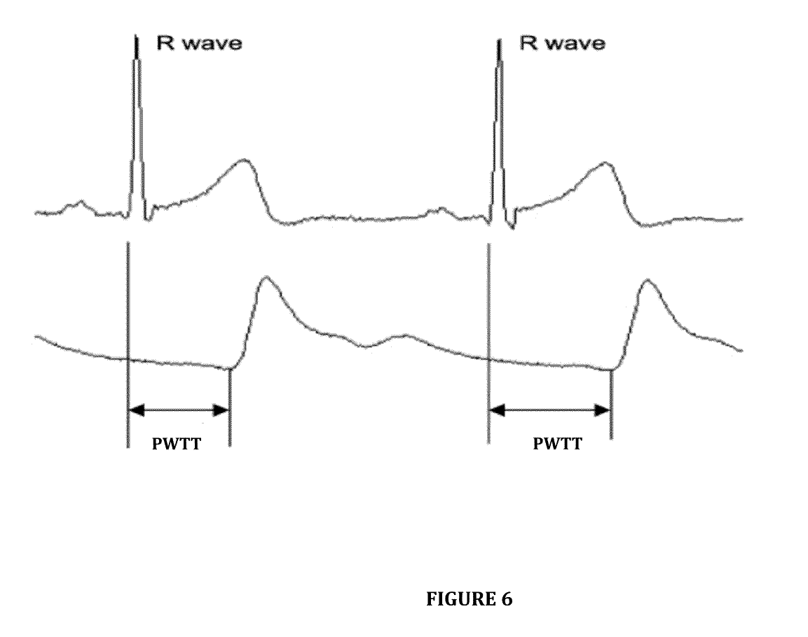

FIG. 6 shows a Pulse Wave Transit Time (PWTT) measured from an ECG waveform and pulse wave using a mobile device that performs mobile cardiac health monitoring in accordance with some embodiments. Referring to FIG. 6, the starting point of PWTT is the peak of R wave on ECG, and there are several different choices for ending point on pulse wave, for example, the foot, peak, or maximum slope point.

In particular, FIG. 6 shows the measurement of PWTT from a simultaneous ECG set of data and pulse wave set of data (e.g., synchronized ECG data and pulse wave data captured using an ECG sensor and an optical sensor, respectively, of the mobile device, such as described herein). In some implementations, a process to determine (e.g., estimate) a measurement of PWTT using a simultaneous ECG and pulse wave includes the following: (1) synchronize ECG and pulse wave detected from the ECG sensor and optical sensor; (2) detect the R-wave peak of ECG; and (3) calculate PWTT. In some embodiments, PWTT is calculated from the time interval between the R-wave peak of the ECG data and pulse wave arrival when the ECG data and pulse wave are simultaneously recorded. In some embodiments, PWTT is the time interval from the R-wave peak to the foot of the pulse wave. In some embodiments, PWTT is calculated from the interval between the R-wave peak and when the differentiated pulse wave reaches, for example, 30% of the peak differentiated pulse wave.

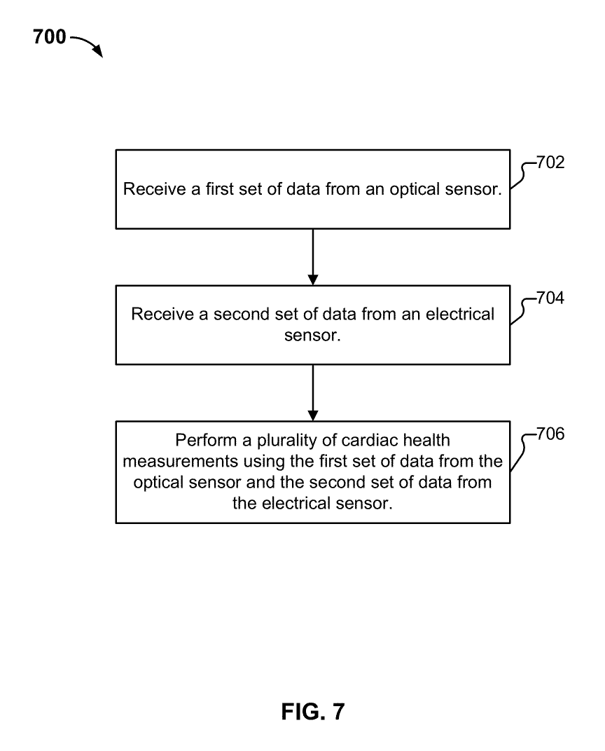

FIG. 7 is a flow diagram for performing mobile cardiac health monitoring in accordance with some embodiments. In some embodiments, process 700 is performed using a mobile device that includes a processor, an optical sensor, and an electrical sensor(s). In some embodiments, the electrical sensor(s) can be integrated in a case for the mobile device. In some embodiments, the electrical sensor(s) can be integrated with the mobile device. At 702, receiving a first set of data from an optical sensor is performed. At 704, receiving a second set of data from an electrical sensor is performed. At 706, performing a plurality of cardiac health measurements using the first set of data from the optical sensor and the second set of data from the electrical sensor. In some embodiments, the electrical sensor includes an electrocardiography (ECG) sensor(s). In some embodiments, the processor is further configured to control a resolution of the optical sensor (e.g., such as 720×480 pixel resolution). In some embodiments, the processor is further configured to control a sampling rate of the optical sensor (e.g., such as to use a sampling rate of 30 Hertz (Hz) or higher). In some embodiments, a plurality of cardiac health measurements includes ECG, heart rate, blood pressure, and cardiac output.

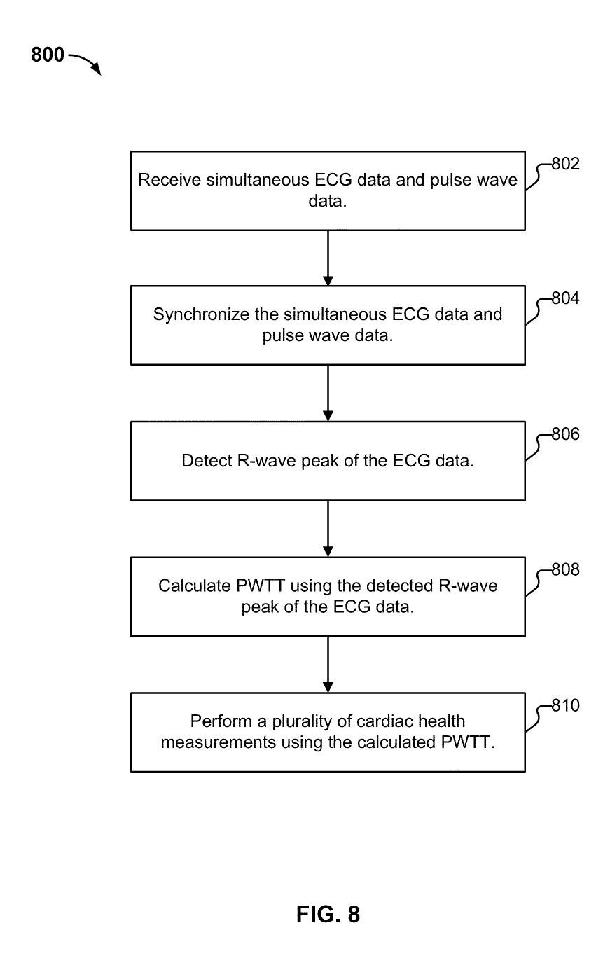

FIG. 8 is another flow diagram for performing mobile cardiac health monitoring in accordance with some embodiments. In some embodiments, process 800 is performed using a mobile device that includes a processor, an optical sensor, and an electrical sensor(s). In some embodiments, the electrical sensor(s) can be integrated in a case for the mobile device. In some embodiments, the electrical sensor(s) can be integrated with the mobile device. At 802, simultaneous ECG data and pulse wave data is received (e.g., the simultaneous ECG data and pulse wave data can be measured using an ECG sensor and an optical sensor, respectively, of a mobile device and/or such sensors can be integrated in a case for the mobile device). At 804, the simultaneous ECG data and pulse wave data is synchronized. At 806, the R-wave peak of the ECG data is detected. At 808, PWTT is calculated using the detected R-wave peak. In some embodiments, PWTT is calculated from the time interval between the R-wave peak of ECG and pulse wave arrival when ECG and pulse wave are simultaneously recorded. In some embodiments, PWTT is calculating from the time interval from the R-wave peak to the foot of pulse wave. In some embodiments, PWTT is calculated from the interval between the R-wave peak and when the differentiated pulse wave reaches, for example, 30% of the peak differentiated pulse wave. At 810, a plurality of cardiac health measurements are performed using the calculated PWTT.

The calculated PWTT can be used to determine various cardiac health measurements. For example, the calculated PWTT can be used as an indirect estimation of blood pressure of the user holding the mobile device. As another example, the calculated PWTT can be used to provide an estimate of cardiac output. In some embodiments, as shown in and described above with respect to FIG. 3, for example, a user places one of his/her fingers on the lens of the camera of smart phone, then the image or a portion of the image, for example, a grayscale portion of the image, is scanned and processed, resulting in brightness information for every frame. Every heart beat creates a wave of blood that reaches the capillaries in the tip of the finger. When capillaries are full of blood, they generally will obstruct the light resulting in lower average brightness values. As blood is retraced, more light can pass through resulting in higher average brightness. By this way, pulse wave is captured by extracting, for example, the average brightness values for each frame. During this process, ECG can be simultaneously captured by placing two hands on ECG electrodes. The data can be aligned with each other, for example by timestamps of video and ECG signals. To measure PWTT, R-wave peak detection from the ECG signal, beat-beat detection, and a particular point detection of pulse wave, such as the foot point of pulse wave are performed. Many techniques have been derived to characterize the relationship between PWTT and blood pressure and cardiac output (e.g., such as an overall blood pressure (BP) was approximated by,

$$BP = \frac{A}{{PWTT}^{ 2}} + B ,$$

as described in publication of P. Fung et al. “Continuous Noninvasive Blood Pressure Measurement by Pulse Transit Time”, Proceedings of the 26th Annual International Conference of the IEEE EMBS, San Francisco, Calif., September, 2004. A is estimated from subject height,

$$A = {( 0.6 \times height )}^{2} \times \frac{ρ}{1.4} .$$

B is a calibration value. Cardiac output (CO) can be derived as CO=K×(α×PWTT+β)×HR as described in H. Ishihara, et al. “A New Non-invasive Continuous Cardiac Output Trend Solely Utilizing Routine Cardiovascular Monitors”, Journal of Clinical Monitoring and Computing, 18: 313-320, 2004, where HR represents heart rate and K, α, and β can be obtained through calibration. In addition to estimate blood pressure and cardiac output, other physiological parameters also can be monitored by the system, such as heart rate, heart rate variability, and respiration.

Although the foregoing embodiments have been described in some detail for purposes of clarity of understanding, the invention is not limited to the details provided. There are many alternative ways of implementing the invention. The disclosed embodiments are illustrative and not restrictive.

What is claimed is:

1. A system for mobile cardiac health monitoring, comprising:
a processor of a mobile device, wherein the processor is configured to:
receive a first set of data from an optical sensor;
receive a second set of data from an electrical sensor; and
perform a plurality of cardiac health measurements using the first set of data from the optical sensor and the second set of data from the electrical sensor; and
a memory coupled to the processor and configured to provide the processor with instructions.

2. The system recited in claim 1, wherein the electrical sensor includes an electrocardiography (ECG) sensor, and wherein the plurality of cardiac health measurements includes one or more of following: ECG, heart rate, blood pressure, and cardiac output.

3. The system recited in claim 1, wherein the electrical sensor is integrated in a case for the mobile device.

4. The system recited in claim 1, wherein the electrical sensor is integrated with the mobile device.

5. The system recited in claim 1, wherein the processor is further configured to:
control a resolution and sampling rate of the optical sensor.

6. The system recited in claim 1, wherein the processor is further configured to:
determine a blood pressure and a cardiac output related index of a user using the first set of data from the optical sensor and the second set of data from the electrical sensor.

7. The system recited in claim 1, wherein the processor is further configured to:
determine a Pulse Wave Transit Time (PWTT) using the first set of data from the optical sensor and the second set of data from the electrical sensor.

8. The system recited in claim 1, wherein the first set of data detected from the optical sensor includes pulse wave data, wherein the second set of data detected from the electrical sensor includes electrocardiography (ECG) data, and wherein the processor is further configured to:
determine a Pulse Wave Transit Time (PWTT) using the pulse wave data and the ECG data.

9. The system recited in claim 1, wherein the first set of data detected from the optical sensor includes pulse wave data, wherein the second set of data detected from the electrical sensor includes electrocardiography (ECG) data, and wherein the processor is further configured to:
receive simultaneous ECG data and pulse wave data;
synchronize the ECG data and the pulse wave data; and
determine a Pulse Wave Transit Time (PWTT) using the ECG data and the pulse wave data.

10. The system recited in claim 1, wherein the first set of data from the optical sensor includes pulse wave data, wherein the second set of data from the electrical sensor includes electrocardiography (ECG) data, and wherein the processor is further configured to:
receive simultaneous ECG data and pulse wave data;
synchronize the ECG data and the pulse wave data;
detect an R-wave peak of the ECG data; and
calculate a Pulse Wave Transit Time (PWTT) using the detected R-wave peak of the ECG data.

11. A method for mobile cardiac health monitoring, comprising:
receiving a first set of data from an optical sensor of a mobile device;
receiving a second set of data from an electrical sensor; and
performing a plurality of cardiac health measurements using the first set of data from the optical sensor and the second set of data from the electrical sensor.

12. The method of claim 11, wherein the electrical sensor includes an electrocardiography (ECG) sensor, and wherein the plurality of cardiac health measurements includes ECG, heart rate, blood pressure, and cardiac output.

13. The method of claim 11, wherein the electrical sensor is integrated in a case for the mobile device.

14. The method of claim 11, wherein the electrical sensor is integrated with the mobile device.

15. The method of claim 11, further comprising:
controlling a resolution and sampling rate of the optical sensor.

16. A computer program product for mobile cardiac health monitoring, the computer program product being embodied in a tangible computer readable storage medium and comprising computer instructions for:
receiving a first set of data from an optical sensor of a mobile device;
receiving a second set of data from an electrical sensor; and
performing a plurality of cardiac health measurements using the first set of data from the optical sensor and the second set of data from the electrical sensor.

17. The computer program product recited in claim 16, wherein the electrical sensor includes an electrocardiography (ECG) sensor, and wherein the plurality of cardiac health measurements includes ECG, heart rate, blood pressure, and cardiac output.

18. The computer program product recited in claim 16, wherein the electrical sensor is integrated in a case for the mobile device.

19. The computer program product recited in claim 16, wherein the electrical sensor is integrated with the mobile device.

20. The computer program product recited in claim 16, further comprising computer instructions for:
controlling a resolution and sampling rate of the optical sensor.
